(19) United States
(12) Patent Application Publication
(10) Pub. No.: US 2005/0095600 A1
(43) Pub. Date: May 5, 2005

(54) Methods of generating gene-specific probes for nucleic acid array detection

(76) Inventors: Xiang Yu, Germantown, MD (US); Li Shen, Boyds, MD (US); Xiao Zeng, Frederick, MD (US); Ying Han, Germantown, MD (US); Kun Lu, Gaithersburg, MD (US); Hui Cen, Oakland, CA (US)

Correspondence Address: MORRISON & FOERSTER LLP, 755 PAGE MILL RD, PALO ALTO, CA, 94304-1018, US

(21) Appl. No.: 10/700,304

(22) Filed: Oct. 31, 2003

(51) Int. Cl. C12Q 1/68; C12P 19/34

(52) U.S. Cl. 435/6; 435/91.2

(57) ABSTRACT

This invention provides gene-specific probe labeling methods for nucleic acid array detection. In the subject invention, a set of a representational number of distinct gene specific primers is used to generate a sub-population of labeled nucleic acids from each of the different physiological samples. The labeled nucleic acids are then compared to each other by hybridizing to a specially designed nucleic acid array that contains the representational number of distinct genes. The subject methods find use in identifying the expression pattern of the genes of special interest in the physiological samples.

### BACKGROUND OF THE INVENTION

1. Technical Field

The technical field of this invention is analysis of gene expression using nucleic acid arrays (cDNA array, gene array, gene chip, or microarray).

2. Background Art

Since its introduction in the 1990's (Schena M et al., Trends Biotechnol. (1998) 16:301-306, and Ekins R and Chu F W, Trends Biotechnol. (1999) 17:217-218), nucleic acid array technology has dramatically changed the way that researchers approach many biomedical subjects. Nucleic acid arrays allow researchers to measure the expression levels of thousands of genes simultaneously. When these profiles of gene expression are compared among experimental or clinical conditions, they can provide extensive information on gene interaction and function. Further analysis of this information has allowed researchers to categorize many subtypes of cancers that were impossible by the traditional methods (Chung C H et al., Nat Genet (2002) Suppl:533-40; Ramaswamy S et al., Proc Natl Acad Sci USA (2001) 98(26):15149-54; Brenton J D et al., Breast Cancer Res (2001) 3(2):77-80). It also has provided a powerful tool for identifying novel molecular drug targets that may have the potential to bring the cure for various types of diseases (Davis R E and Staudt L M., Curr Opin Hematol (2002) 9(4):333-8.). One of the recent trends in array technology is the emergence of application-specific nucleic acid arrays (Fitzgerald D A and Guimbellot J S., The Scientist (2001) 15(18):26, and Constans A., The Scientist (2003) 17(3):35). Contrary to the global nucleic acid arrays, these application-specific arrays have a build-in knowledge in choosing and grouping the genes that are especially important to certain biological pathways. Besides its appeal of low cost, this type of arrays has the potential to deliver more sensitive and reliable data.

In order to get the signal readout from a nucleic acid array, expressed genes (in the form of RNAs called mRNA) have to be converted to detectable molecules that are either radioactive or fluorescent. Alternatively, mRNAs could be converted to molecules that contain a moiety that generates fluorescent or chemiluminescent signals when contacting appropriate substrates. In general practice, random oligonucleotides, oligo-dT and/or gene specific primers (Maniatis et al., Molecular Cloning: A Laboratory Manual. Cold Spring Harbor Press, 1989, U.S. Pat. No. 6,352,829, U.S. Appl. Pub. No. 20010007744) are used to prime enzymatic reaction that converts mRNA to labeled cDNA through reverse transcription. An unexpected drawback for this labeling method is the non-specific probe labeling caused by endogenous RNA self-priming. Since mRNA comprises only a small fraction of total RNA (˜5%), a large portion of the labeled cDNA by this direct RT-labeling method is not the product of the poly-A containing or gene-specific primer targeted mRNA. Consequently, the background level of the array readout is usually high, and sometimes false-positive results are produced. Although increasing reaction temperature (Clontech, Ambion) or using a special reaction buffer and/or mutant reverse transcriptase (Ambion, Invitrogen, SuperArray) may decrease the nonspecific endogenous priming, neither method can eliminate RNA endogenous priming entirely. In addition, since the reverse transcriptase reaction is normally done in an isothermal environment, multiple priming events (especially for specific primers) on the same RNA template are rare, therefore no amplification of the initial mRNA can be achieved through the labeling RT reaction.

Recently, an invention has been proposed to amplify RNA extracted from as little as a single cell (Eberwine J H et al., Proc Natl Acad Sci USA (1992) 89:3010-3014; Phillips J and Eberwine J H., Methods. (1996) 10 (3):283-8; and U.S. Pat. Nos. 5,932,451 and 5,545,522) without skewing the gene expression profile using T7 RNA polymerase mediated antisense RNA (aRNA) amplification. This invention has made the tool of nucleic acid array available to clinicians who were limited by the shortage of patient samples or the heterogeneity of the biopsies. However, little attention has been drawn to the fact that amplifying the whole population of mRNA does not necessarily allow the low abundant messages to be detected by the array because they do not have any gain proportionally compared to other mRNAs in the population.

PCR technology has been effectively used in gene expression analysis in many different ways such as subtractive cloning for cDNA library construction (reviewed by Sagerström C G et al., Annu. Rev. Biochem. (1997) 66:751-783), differential display RT-PCR (DDRT-PCR) (Liang P and Pardee A B., Science. (1992) 257(5072):967-71.), and 3 prime end amplification (TPEA) PCR (Dixon A K et al., Nucleic Acids Res. (1998) 26(19):4426-31.). However, there has been some reluctance in using PCR-based amplification methods in array analysis, because users fear that the exponential nature of the PCR amplification generates biased profile of the starting RNA samples (Baugh L R et al., Nucleic Acids Res. (2001) 29(5):E29). Recently, using a so called “global cDNA amplification” protocol, Iscove and colleagues showed that the exponentially amplified cDNA still conserved its original composition even after 90 cycles of PCR (Iscove et al., Nat Biotechnol. (2002) 20(9):940-3; also see Aoyagi K et al., Biochem Biophys Res Commun. (2003) 300(4):915-20.). Clontech's SMART™ cDNA technology (BD Biosciences, formerly known as CapFinder: http://www.clontech.com/archive/JAN96UPD/CapFinder.shtml) is another version of this PCR-based global mRNA amplification method (SMART stands for Switching Mechanism At 5′ end of RNA Template). Again, in these “global” amplification schemes, the low abundant messages would remain under-represented after the “global” probe labeling reaction.

Despite the promise of the nucleic acid array technology, there are many aspects of the technology that need to be improved. The subject invention addresses the probe labeling process, one of the key steps that affect the sensitivity and reliability of the technology. In accordance with the concept of the newly developed application-specific nucleic acid arrays, the subject invention provides a fast, simple and reliable gene-specific probe labeling method that greatly improves the sensitivity and reliability of nucleic acid array detection.

### SUMMARY OF THE INVENTION

The invention disclosed herein concerns methods of generating a sub-population of labeled nucleic acids for use in profiling gene expression with nucleic acid array technology.

In one aspect, this invention provides a method of producing a sub-population of labeled nucleic acids, said method comprising: (a) synthesizing first strand cDNA from a sample of RNA through reverse transcription, wherein the sample of RNA is obtained from a physiological source; (b) contacting said first strand cDNA with a pool of a representational number of at least 15 distinct gene specific primers under conditions that allow formation of hybrid duplexes between said gene specific primers and said first strand cDNA, wherein each constituent gene specific primer has a sequence complementary to a distinct first strand cDNA; and (c) enzymatically extending said gene specific primers from said hybrid duplexes to generate a sub-population of labeled nucleic acids.

In some embodiments, the sub-population of labeled nucleic acids extended from the gene specific primers is generated through a single cycle of uni-directional DNA polymerization. In other embodiments, the sub-population of labeled nucleic acids extended from the gene specific primers is generated through multiple cycles of uni-directional DNA polymerization. In some embodiments, thermophilic enzymes (such as Taq DNA polymerase) are used to enzymatically extend gene specific primers from the hybrid duplexes to generate a sub-population of labeled nucleic acids.

In another aspect, the invention provides a method of producing a sub-population of labeled nucleic acids, said method comprising: (a) synthesizing first strand cDNA from a sample of RNA through reverse transcription, wherein the sample of RNA is obtained from a physiological source; (b) generating a sub-population of labeled nucleic acids using polymerase chain reaction (PCR) with a pool of a representational number of at least 15 pairs of distinct gene specific primers, wherein one gene specific primer in each pair comprises a sequence complementary to the sense sequence of said distinct gene, and the other primer in the pair comprises a sequence complementary to the antisense sequence of said distinct gene.

In some embodiments, asymmetric PCR is used for generating the sub-population of labeled nucleic acids. In some embodiments, the labeled nucleic acids are generated through a single cycle of PCR. In other embodiments, the labeled nucleic acids are generated through multiple cycles of PCR.

In yet another aspect, the invention provides a method of analyzing the differences in the expression pattern of the genes of special interest between a plurality of different physiological samples, said method comprising: (a) synthesizing first strand cDNA from a sample of RNA through reverse transcription, wherein the sample of RNA is obtained from a physiological source; (b) contacting a pool of a representational number of at least 15 distinct gene specific primers with said first strand cDNA under conditions that allow formation of hybrid duplexes between said gene specific primers and said first strand cDNA, wherein each constituent gene specific primer has a sequence complementary to a distinct first strand cDNA; and (c) enzymatically extending said gene specific primers from said hybrid duplexes to generate a sub-population of labeled nucleic acids; and (d) comparing the populations of labeled nucleic acids from each physiological source to identify the differences in the populations.

In yet another aspect, the invention provides a method of analyzing the differences in the expression pattern of the genes of special interest between a plurality of different physiological samples, said method comprising: (a) synthesizing first strand cDNA from a sample of RNA through reverse transcription, wherein the sample of RNA is obtained from a physiological source; (b) generating a sub-population of labeled nucleic acids using polymerase chain reaction (PCR) with a pool of a representational number of at least 15 pairs of distinct gene specific primers, wherein one gene specific primer in each pair comprises a sequence complementary to the sense sequence of said distinct gene, and the other primer in the pair comprises a sequence complementary to the antisense sequence; and (c) comparing the populations of labeled nucleic acids from each physiological source to identify the differences in the populations.

In some embodiments, the comparing step of the invention comprises: hybridizing the labeled nucleic acids from each of the distinct physiological samples to an array of nucleic acids stably associated with the surface of a substrate; washing off the unbound labeled nucleic acids from the surface to produce a detectable hybridization patterns for each of the distinct physiological samples; and comparing the hybridization patterns for each of the distinct physiological samples.

In any of the embodiments described herein, the sample of RNA may be total RNA, mRNA, or amplified antisense RNA (aRNA). The first strand cDNA may be synthesized through RNA self-priming without addition of any exogenous synthetic primers, using synthetic random primers, or using oligo dT primers. The pool of gene specific primers may comprise at least 20 distinct gene specific primers, at least 50 distinct gene specific primers, or at least 100 distinct gene specific primers. The pool of gene specific primers may comprise one or more oligonucleotide sequences for a single gene. The label may be directly detectable or detectable after a subsequent chemical or enzymatic reaction.

### BRIEF DESCRIPTION OF THE DRAWINGS

FIG. 1 shows the hybridization patterns obtained by hybridizing labeled probes generated by MMLV reverse transcriptase method (A) and LPR method described in Example 1 (B) to an array (GEArray™ Q-series Human TGFβ/BMP Signaling Pathway Kit, SuperArray, Cat#HS-023N, generated according to Example 3). The RNA used to generate the labeled probes was Human Universal Reference RNA (BD. Biosciences, Clontech Cat #636538).

FIG. 2 shows the hybridization patterns obtained by hybridizing labeled probes generated by MMLV reverse transcriptase method (A) and LPR method described in Example 1 (B) to an array (GEArray Q-series Human Neurotrophin and Receptors Gene Array, SuperArray, Cat#HS-018, generated according to Example 3). The RNA used to generate the labeled probes was antisense RNA (aRNA) prepared by two rounds of amplification with RiboAmp™ OA RNA Amplification Kit (Arcturus, Cat#KIT0206) from human brain total RNA (BD Biosciences, Clontech Cat#36530).

FIG. 3 shows the hybridization patterns obtained by hybridizing labeled probes generated by MMLV reverse transcriptase method (A), LPR method described in Example 1 (B), and RT-PCR profile (C) to an array (GEArray™ Q-series Mouse Insulin Signal Pathway Kit, SuperArray, Cat#MM-030N, generated according to Example 3). The RNA used to generate the labeled probes was total RNA from mouse liver and thymus (BD Biosciences).

### MODES OF CARRYING OUT THE INVENTION

This invention provides methods of generating a sub-population of labeled nucleic acids used for profiling gene expression with the nucleic acid array technology. In this invention, a set of a representational number of distinct gene specific primers is used to generate a sub-population labeled nucleic acids through a single or multiple cycles of uni-directional DNA polymerization or PCR including asymmetrical PCR (for PCR, at least one pair of gene specific primers is used for each distinct gene) from either total RNA, mRNA or linearly amplified aRNA from physiological samples. Alternatively, the starting material could be an amplified cDNA library that is derived from RNA samples. This invention greatly reduces background (noise) level on the array caused by endogenous RNA self-priming during reverse transcription. In addition, this method allows the investigator to control how many genes are detected on the array. Finally, the number of amplification cycles is also flexible, allowing the investigator to set the detection sensitivity of an array according to the relative expression levels of the distinct gene set interested.

For clarity of disclosure, and not by way of limitation, the detailed description of the invention is divided into the subsections that follow. Before the subject invention is further described, it is to be understood that the invention is not limited to the particular embodiments of the invention described below, as variations of the particular embodiments may be made and still fall within the scope of the appended claims.

A. Definitions

Unless defined otherwise, all technical and scientific terms used herein have the same meaning as is commonly understood by one of ordinary skill in the art to which this invention belongs. All patents, applications, published applications referred to herein are incorporated by reference in their entirety. If a definition set forth in this section is contrary to or otherwise inconsistent with a definition set forth in applications, published applications that are herein incorporated by reference, the definition set forth in this section prevails over the definition that is incorporated herein by reference.

As used herein, “a” or “an” means “at least one” or “one or more.”

As used herein, “nucleic acid (s)” refers to any sized multimer of nucleotide monomeric units in any form, including inter alia, single-stranded, duplex, triplex, linear and circular forms. It includes short multimers such as dimers, trimers, and the like. It also includes polynucleotides, oligonucleotides, chimeras of nucleic acids and analogues thereof. The nucleic acids described herein can be composed of the well-known deoxyribonucleotides and ribonucleotides composed of the bases adenosine, cytosine, guanine, thymidine, and uridine, or may be composed of analogues or derivatives of these bases. Additionally, various other oligonucleotide derivatives with nonconventional phosphodiester backbones are also included herein, such as phosphotriester, polynucleopeptides (PNA), methylphosphonate, phosphorothioate, polynucleotides primers, locked nucleic acid (LNA) and the like.

As used herein, “primer” refers to an oligonucleotide that hybridizes to a target sequence, typically to prime the nucleic acid in the amplification process.

As used herein, “probe” refers to an oligonucleotide that hybridizes to a target sequence, typically to facilitate its detection. The term “target sequence” or “target nucleic acid molecule” refers to a nucleic acid sequence to which the probe specifically binds. Unlike a primer that is used to prime the target nucleic acid in the amplification process, a probe need not be extended to amplify target sequence using a polymerase enzyme. However, it will be apparent to those skilled in the art that probes and primers are structurally similar or identical in many cases.

As used herein, “complementary” refers to the hybridization or base pairing between nucleotides or nucleic acids, such as, DNA:DNA, or DNA:RNA, or RNA:RNA duplex. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, preferably at least about 95%, 96%, 97%, 98%, 99%, or 100%. “Complementary” also means that two nucleic acid sequences can hybridize under low, middle and/or high stringency condition(s). “Substantially complementary” also means that two nucleic acid sequences can hybridize under high stringency condition(s).

As used herein: “stringency of hybridization” is as follows:
1) high stringency: 0.1×SSPE (or 0.1×SSC), 0.1% SDS, 65° C.;
2) medium stringency: 0.2×SSPE (or 1.0×SSC), 0.1% SDS, 50° C. (also referred to as moderate stringency); and
3) low stringency: 1.0×SSPE (or 5.0×SSC), 0.1% SDS, 50° C.

It is understood that equivalent stringencies may be achieved using alternative buffers, salts and temperatures.

As used herein, “gene” refers to the unit of inheritance that occupies a specific locus on a chromosome, the existence of which can be confirmed by the occurrence of different allelic forms. Given the occurrence of split genes, gene also encompasses the set of DNA sequences (exons) that are required to produce a single polypeptide.

B. Sample of RNA and the Synthesis of First Strand cDNA

The first step in the subject methods is to obtain a sample of nucleic acids, usually RNAs (e.g, total RNA, mRNA, aRNA) or nucleic acid derivatives thereof, from a physiological source. Samples of nucleic acids can also be obtained from a plurality of physiological sources, where the term plurality is used to refer to 2 or more distinct physiological sources. The physiological source of RNAs may be eukaryotic, with physiological sources of interest including sources derived single celled organisms such as yeast and multicellular organisms, including plants and animals, particularly mammals (including humans), where the physiological sources from multicellular organisms may be derived from particular organs or tissues of the multicellular organism, or from isolated cells derived therefrom. Thus, the physiological sources may be different cells from different organisms of the same species, e.g. cells derived from different humans, or cells derived from the same human (or identical twins) such that the cells share a common genome, where such cells will usually be from different tissue types, including normal and diseased tissue types, e.g. neoplastic cell types. In obtaining the sample of RNAs to be analyzed from the physiological source from which it is derived, the physiological source may be subjected to a number of different processing steps, where such processing steps might include tissue homogenation, nucleic acid extraction and the like, where such processing steps are known to the those of skill in the art. For example, methods of isolating RNA from cells, tissues, organs or whole organisms are described in Maniatis et al., Molecular Cloning: A Laboratory Manual (Cold Spring Harbor Press)(1989). Antisense RNA (aRNA) may be synthesized from total RNA using methods known in the art. mRNA may also be isolated from total RNA using methods known in the art.

In the subject method, the next step is to convert RNA (for example, total RNA, mRNA or aRNA) into first strand cDNA. RNA is usually converted into first strand cDNA enzymatically by reverse transcriptase (RT), a RNA-dependent DNA polymerase. Exemplary reverse transcriptase (RT) includes, but not limited to, the Moloney murine leukemia virus (M-MLV) RT as described in U.S. Pat. No. 4,943,531, a mutant form of M-MLV-RT lacking Rnase H activity as described in U.S. Pat. No. 5,405,776, human T-cell leukemia virus type I (HTLV-I) RT, bovine leukemia virus (BLV) RT, Rous sarcoma virus (RSV) RT, Avian Myeloblastosis Virus (AMV) RT and human immunodeficiency virus (HIV) RT. RTs suitable for this purpose may also be extracted from their natural hosts. Alternatively, RTs can be obtained commercially or isolated from host cells that express high levels of recombinant forms of the enzymes by methods known to those of skill in the art. The particular manner of obtaining the reverse transcriptase may be chosen based on factors such as convenience, cost, availability and the like.

Reverse transcriptase can extend the free end of an oligonucleotide (or a primer) that forms a stable base pairing with the target RNA molecule. Under most conditions, RT enzymes can produce cDNA molecules without the, supply of exogenous primers (http://www.superarray.com/gene_array_protocol/trueLabel.pdf). Alternatively, exogenous primers may be used. Two types of exogenous primers, random primers and specific primers, may be added to the reaction to facilitate the cDNA synthesis. Random primers, which have defined length but no defined sequence, can be used to prime the conversion of RNA to cDNA without discrimination of RNA species. The length of random primers is usually between 2 and 25 nucleotides (nt), but more often between 5 and 10 nt. The most commonly used is the random hexamers (6 nt). Specific primers, which have defined length and defined nucleotide sequence, may also be used to synthesize cDNA from a defined sub-population of RNA. An example of such specific primers is the oligo dT primers. The length of the oligo dT primers may be between 10 to 40 nt, between 15 and 25 nt, or about 18 nt. The oligo dT primers selectively anneal with RNA that contains a poly-A tail at its 3′ end, a characteristic of most messenger RNA in cells. Another example of a specific primer is the gene-specific primer. A gene-specific primer may have a sequence complementary to that of a distinct RNA. Preferably, the gene-specific primer has a sequence substantially complementary to that of a distinct RNA. In some embodiments, a gene-specific primer primes the synthesis of cDNA from one unique sequence within a RNA molecule that corresponds to one single gene.

Exogenous primers can be synthesized according to conventional oligonucleotide chemistry methods, in which the nucleotide units may be: (A) solely nucleotides found in naturally occurring DNA and RNA, e.g., adenine, cytosine, guanine, thymine and uracil; or (B) solely nucleotide analogs that are capable of base pairing under hybridization conditions in the course of DNA synthesis such that they function as the nucleotides described in (A), e.g., inosine, xanthine, hypoxanthine, 1,2-diaminopurine and the like; or (C) any combination of the nucleotides described in both (A) and (B).

The buffer necessary for first strand cDNA synthesis may be purchased commercially from various sources, such as SuperArray, Promega, Invitrogen, Clontech, Amersham. These buffers have a pH ranging from 6 to 9, with 10-200 mM of Tris-HCl or HEPES. Other salts may include NaCl, KCl, MgCl₂, Mg (OAc)₂, MnCl2, Mn(OAc)₂ etc., at concentrations ranging from 1-200 mM. Additional reagents such as reducing agents (DTT), detergents (TritonX-100), albumin and the like may be supplemented in the buffer. Chemical compound or polymers, such as DMSO, poly-lysine, betaine, and the like, may be added to the buffer to prevent RNA from forming secondary structures. Depending on the particular nature of the assay, a combination of the above mentioned reagents might be chosen to limit endogenous priming during reverse transcription.

Deoxyribonucleoside triphosphates (dNTPs) necessary for first strand cDNA synthesis through reverse transcription of RNAs may be purchased commercially from various sources, such as SuperArray, Promega, Invitrogen, Clontech, Amersham. In the reaction, dNTPs may comprise: (A) only the nucleotides that are commonly found in DNA, e.g. dATP, dGTP, dCTP dTTP and dUTP; or (B) analogs of above mentioned nucleotides that are less frequently found in nature, such as those with ribose moieties like inosine, xanthine, hypoxanthine; or (C) any combination of the nucleotides described in (A) and (B). The use of the combination of nucleotide analogs may be helpful separating the newly synthesized DNA from the genomic DNA that may co-purify with the total RNA. Derivatives of inosine are an example commonly used for this purpose. Newly synthesized dITP-containing DNA has a lower melting temperature (Tm) than the corresponding natural DNA, such as genomic DNA (Auer et al. Nucleic Acids Res. 1996;24:5021-5025, U.S. Pat. No. 5,618,703), and (Levy D D and Teebor G W. Nuc. Acids Res. 1991;19(12):3337-3343; Warren R A. Annu. Rev. Microbiol. 1980;34:137-158). Another unconventional nucleotide affecting the Tm of the newly synthesized DNA product is hydroxymethyl dUTP (HmdUTP), which occurs naturally in phage SP01 genomic DNA in place of dTTP The Tm of HmdUTP-containing DNA is 10° C. lower than normal DNA. (Levy D D and Teebor G W. Nuc. Acids Res. 1991;19(12):3337-3343).

C. Annealing a Pool of Representational Number of Distinct Gene-Specific Primers to the First Strand cDNA

The invention uses a set of a representational number of gene-specific primers to generate a sub-population of labeled nucleic acids probes from the first strand cDNA molecules.

As used herein, a “representational number of primers” indicates that the total number of genes represented is only a fraction of the total number of distinct RNA molecules in the physiological sample. For example, the total number of primers is less than 80%, less than 50%, less than 20%, less than 10% of the total number of distinct RNAs, usually the total number of distinct mRNAs in the sample. In the subject invention, any two RNA molecules in a sample is considered distinct or different if one of them contains a stretch of at lease 100 nucleotides that is not shared by the other with a sequence similarity over 95%, as determined by the BLAST algorithm (default settings). By this definition, any RNA sample from a physiological source normally contains 5,000 to 50,000 distinct RNA molecules. In the subject invention, the number of gene specific primers in a given set may range from 20 to 20,000, from 50 to 5,000, from 50 to 2,000, or from 15 to 1500. In the subject invention, any two primers are considered distinct or different if their sequence homology is less than 95%, as determined by the BLAST algorithm (default settings). In a particular primer set, one or more primers may represent one distinct RNA molecule at different sequence regions.

Each of the gene specific primer in the set is usually long enough to specifically anneal to a distinct sequence of a first strand cDNA molecule. The length of the gene specific primers may range from 10 to 40 nt, more usually from 12 to 30 nt and most usually from 15 to 25 nt. Generally, the gene specific primers is sufficiently specific to hybridize to complementary template sequence (the first strand cDNA) during the generation of labeled nucleic acids under conditions sufficient for DNA synthesis, which conditions are known by those of skill in the art. In some embodiments, as determined by BLAST algorithm (default setting), the number of mismatches between the gene specific primer and its target first strand cDNA generally does not exceed 20%, more usually does not exceed 10% and most usually does not exceed 5%.

In some embodiments, for generating probes to be hybridized to an array, the sequence of each gene specific primer in the set is carefully chosen according to one or more of the following criteria: (A) The extended product c DNA from the primer shares at least 50 nt, at least 100 nt, or at least 200 nt sequence with the nucleic acid fragment immobilized on the array; (B) the Tm and other characteristics of the different gene-specific primers in the same set is similar enough so that their priming efficiency is comparable in the chosen experimental conditions; and (C) the sequence of any gene-specific primer is not prone to form stable secondary structures (with ΔG less than −10 kcal/mol in the default solution), does not comprise stretches of more than 5 identical nucleotides, does not comprise more than 3 repetitive sequences, and/or does not have GC content higher than 80% or lower than 30%.

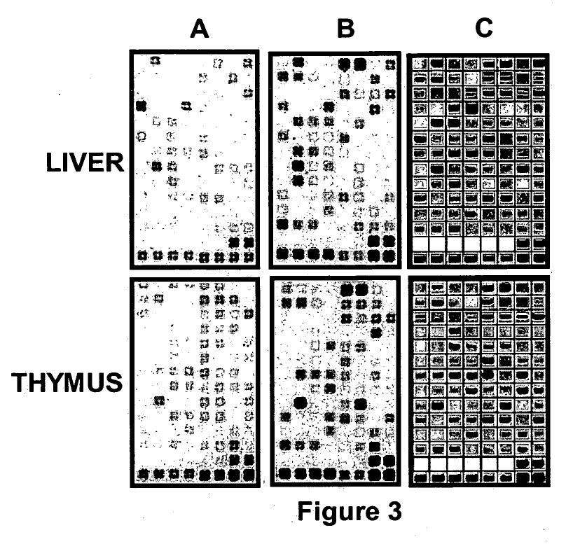

In some embodiments, the number of gene-specific primers in a given set may be the same as the number of genes printed on the nucleic acid array. In some embodiments of the invention, genes of interest are grouped in functional classes or biological pathways. These genes are typically differentially expressed in different cell types (FIG. 3 of Example 5), in disease states, or expressed in response to the influence of external agents, factors or infectious agents, and the like. Preferably, a functional class or a biological pathway is represented in a carefully chosen set of gene-specific primers. Alternatively, more than one (e.g., at least 2, at least 3, at least 4, or at least 5) functional related class (pathway) can be represented in one set of gene specific primers. Examples of gene functional classes are: oncogenes, tumor suppressor genes, cell cycle regulatory genes, stress responsive genes, apoptosis related genes, DNA synthesis/recombination/repair genes, ion channel genes, transporter genes, intracellular signal transduction genes, transcription factor genes, DNA-binding protein genes, receptors genes (including receptors for growth factors, chemokines, interleukins, interferons, hormones, neurotransmitters, cell surface antigens, cell adhesion molecules, etc.), intercellular communication protein genes (such as growth factors, cytokines, chemokines, interleukins, interferons, hormones, etc.), and the like. Gene functional pathways of interest include: the mitogenic pathway, NFκB pathway, p53 pathway, stress response pathway, survival pathway, Wnt pathway, Hedgehog pathway, PKC pathway, p44/42 MAP kinase pathway, p38 MAP kinase pathway, JNK pathway, CREB pathway, PI-3 kinase/Akt pathway, JAK/Stat pathway, TGFβ pathway, BMP pathway, NFAT pathway, insulin pathway, G-protein coupled receptor signaling pathway, and the like. Of particular interest are those gene-specific primers corresponding to the functional classes/pathways listed in the catalog of SuperArray Bioscience Corporation (Frederick, Md.).

In some embodiments, the particular class of genes represented in a chosen set of gene-specific primers reflects the nature of the physiological sources from which the RNAs to be analyzed are derived. For analysis of gene expression profiles of physiological sources, the gene-specific primers usually correspond to the Class II genes, which are transcribed into mRNA molecules with a 5′ cap and a 3′ polyA tail. However, the subject invention does not exclude applications in which the primary interest of the investigation is to detect the presence of foreign organisms in the physiological sample. For example, a clinical sample could be taken to test for infectious agents. In such cases, the chosen set of gene-specific primers would contain those that match the suspected viral, bacterial and/or yeast genes.

Each gene-specific primer may be synthesized by conventional oligonucleotide chemistry methods, where the nucleotide units may be: (A) solely nucleotides found in naturally occurring DNA and RNA, e.g., adenine, cytosine, guanine, thymine and uracil; or (B) solely nucleotide analogs that are capable of base pairing under conditions in the course of DNA synthesis such that they function as the nucleotides described in (A), e.g., inosine, xanthine, hypoxanthine, and the like; or (C) any combination of the nucleotides described in both (A) and (B). The reason for the variety of choices has been discussed previously.

The sets of gene specific primers may comprise primers that correspond to at least 15, at least 20, at least 50, at least 75, at least 100, at least 125, at least 150 distinct genes as represented by distinct mRNAs in the sample. Such sets of gene specific primers are known in the art, and are also described in U.S. Pat. Nos. 5,994,076 and 6,352,829.

D. Generating a Labeled Sub-Population of Nucleic Acid Probes

In the subject method, nucleic acid probes are labeled during synthesis by the incorporation of labeling moieties into newly synthesized DNA in a reaction utilizing a DNA-dependent DNA polymerase. To generate labeled nucleic acid probes, either the gene-specific primers or one of the substrate dNTPs contain a labeling moiety. A labeling moiety is an entity comprising a member of a signal producing system and is detectable, either directly or through combined action with one or more additional members of a signal producing system. When additional members of this signal producing system were to be subsequently reconstituted, a signal can be recorded and the signal intensity can be linearly correlated with the amount of labeling moiety captured. Examples of directly detectable labeling moieties include the dNTPs modified with radioactive isotope or fluorescent dyes. Isotopic moieties include ³²P, ³³P, ³⁵S, ¹²⁵I and the like. Fluorescent moieties include coumarin and its derivatives, such as 7-amino-4-methylcoumarin and aminocoumarin; bodipy dyes, such as Bodipy FL; cascade blue; fluorescein and its derivatives, such as fluorescein isothiocyanate and Oregon green; rhodamine dyes, such as texas red and tetramethylrhodamine; eosins and erythrosins; cyanine dyes, such as Cy3 and Cy5; macrocyclic chelates of lanthanide ions, such as quantum dye; fluorescent energy transfer dyes, such as thiazole orange-ethidium heterodimer, TOTAB, etc. Besides directly detectable labeling moieties, labeling moiety may be a member of a signaling producing system that act in concert with one or more additional members to produce the detectable signal. Examples of such indirect labeling moieties are biotin, digoxigenin, fluorescein, antigen, polyvalent cations, chelator groups and the like. After moiety-modified dNTPs are incorporated in the newly synthesized DNA, additional members of the signal producing system will be provided to finish the detection. For example, after biotinated dUTP is incorporated into newly synthesized DNA and immobilized onto a nylon membrane, the DNA can bind to the streptavidin-conjugated alkaline-phosphatase and produce chemiluminescent signal in-situ when a proper substrate (e.g., CDP-STAR) is added. Alternatively, dNTPs modified with chemical reactive moieties can be incorporated in newly synthesized DNA, and the labeling can be accomplished by a post-synthesis chemical reaction. A preferred example of this type is the use of amino-amyl-dUTP in the DNA synthesis reaction and the use of thio-biotin for post-synthesis labeling (Xiang C C et al. Nat Biotechnol. 2002 July;20(7):738-42.). For each sample of RNA, one can generate labeled oligos with the same labels. Alternatively, one can use different labels for each physiological source, which provides for additional assay configuration possibilities, as described in greater detail below.

DNA-dependent DNA polymerase is used to incorporate the labeling moiety into newly synthesized DNA, or labeled nucleic acid probe. In some embodiments, the DNA polymerases are the thermophilic enzymes such as the Taq DNA polymerase (from Thermus aquaticus), Tth DNA polymerase (from Thermus thermophilus), pfu DNA polymerase, etc. The DNA polymerases should be able to utilize labeling moiety-modified dNTP derivatives as substrate effectively. Suitable DNA polymerase can be obtained commercially or it can be purified from cells that express high levels of the recombinant polymerase by the method known to those of skill in the art.

In some embodiments, before the addition of gene-specific primers and the labeling moieties into reaction buffer, the newly synthesized first strand cDNA is incubated at elevated temperature to inactivate the reverse transcriptase and degrade the RNA template. The temperature of this treatment is usually between 70 and 95° C., preferably no higher than 90° C. and more preferably no higher than 85° C. Depending on the particular nature of dNTPs used in the first strand synthesis, even lower temperature can be used for this treatment. RNase H1 can also be used to facilitate the removal of RNA from first strand cDNA. Alternatively, chemicals such as NaOH can be used to degrade RNA. If chemicals are used, steps must be taken to neutralize and/or remove the chemicals because they may interfere with the subsequent enzymatic labeling process.

In the labeling process of the invention, the first strand cDNA molecules are combined with the set of gene-specific primers, dNTPs and the derivative modified with labeling moiety, and the DNA polymerase. The reaction buffer system and other components are optimized to promote the maximum incorporation of labeling moiety into newly synthesized DNA. The temperatures for the thermal cycling reactions may range between 42 and 85° C. At the lower end temperature, for example, the gene-specific primers will be given enough time to anneal to the target first strand cDNA. At 72° C., for example, the extension of the primers will allow the incorporation of labeling moieties and extension of labeled DNA probes.

In one embodiment, the process for the generation of the labeled nucleic acid DNA comprises one cycle of the enzymatic amplification step. In other embodiments as shown in Examples 1 and 2, the process for the generation of the labeled nucleic acid DNA comprises more than one cycles of the same enzymatic amplification steps including annealing of the gene specific primers to the first strand cDNA and synthesizing the labeled probes. In some of these embodiments, the first strand cDNA molecules are generated by reverse transcription using synthesized random hexamers. The number of thermal cycles used in the labeling process may be 5 or greater, such as 10, 15, 20, 25, or 30. In these embodiments, the direction of the gene-specific primers in the same set usually complementary to the first-strand cDNA for the genes that they represent. In one embodiment, one distinct primer for one target gene is used. In another embodiment, two or more distinct primers can be used for one target gene. When the RNA sample is total RNA or mRNA, the direction of the gene-specific primers in the same set is complementary to the anti-sense sequence of the genes they represent. Yet in another embodiment, when the RNA sample is an anti-sense RNA, the direction of the gene-specific primers in the same set is complementary to the sense sequence of the genes they represent. In some embodiments, primers substantially complementary to the sense or anti-sense sequence of the genes are used. Since the amplification of the labeled DNA is largely linear within the range tested, this method is called Linear Polymerase Replication (LPR).

Preferably, excess amount of gene-specific primers should be used to minimize the competition between gene specific primers and newly synthesized second strand cDNA (the labeled probes). In general, the competition is more significant if high cycle number is used and if the target gene is an abundant gene. In other words, the low abundant genes may benefit if higher cycle number is used.

PCR amplification methods may also be used to generate a sub-population of labeled nucleic acids. For PCR amplification, a pair of gene-specific primers is used to represent a distinct gene. Two primers in each pair are pointing toward each other, with one complementary to the sense sequence of the gene and the other complementary to the antisense strand. By adjusting the relative amount of the two primers, an asymmetric PCR amplification condition can be achieved for the labeling process (Millican D S and Bird I M. Analytical Biochemistry 1997;249:114-17).

In another embodiment, polymerase chain reaction (PCR), as described in U.S. Pat. No. 4,683,195, may be used to produce non-linearly amplified numbers of labeled DNA probes. In the primer set, a pair of gene-specific primers (one sense and the other antisense) may be used to amplify a target gene. The set of these pairs of gene-specific primers may be used to amplify a sub-population of labeled DNA probes, where the PCR conditions may be modified as described in U.S. Pat. No. 5,436,149.

E. Hybridization of the Labeled Probes to Nucleic Acid Arrays

In the subject invention, the labeled nucleic acid probes produced above provide a representative sub-population of the total number of distinct RNA species in the sample. Accordingly, the labeled nucleic acid probes can be used in identifying differences in gene expression among different physiological sources. In one preferable embodiment, one may hybridize the labeled nucleic acid probes to a nucleic acid array where polymeric molecules are immobilized onto the surface of a substrate that are capable of sequence specific base-pairing (hybridization) with the labeled nucleic acid probes.

To analyze differences in gene expression profiles, RNA samples from two or more different physiological sources are converted to labeled DNA probes separately. The labeled DNA probes from each physiological source are incubated with identical nucleic acid arrays, preferably under stringent hybridization conditions, allowing the labeled probes to hybridize specifically to their immobilized complementary nucleic acid molecules on the substrate surface. Alternatively, labeled nucleic acid probes from two physiological sources may be mixed and hybridize to the same nucleic acid array, as long as the two probes can be distinguished by different labeling moieties. Suitable hybridization conditions are well known to those of skill in the art.

Following hybridization, non-hybridized labeled nucleic acid probe is removed from the array surface, conveniently by washing, generating a pattern of hybridized nucleic acid on the substrate surface. A variety of wash conditions is known to those of skill in the art. The resultant hybridization patterns of labeled nucleic acids may be visualized or detected by a variety of ways, depending on the particular labeling moiety incorporated in the labeled nucleic acid probes. Representative detection means include autoradiography, fluorescence measurement, chemiluminescence measurement, colorimetric measurement, light emission measurement and the like. Following detection or visualization, the hybridization patterns may be compared to identify differences between the patterns. Any discrepancies of hybridization signal can be related to a differential expression of a particular gene in the physiological sources being compared.

A variety of different arrays that may be used are known in the art. The polymeric molecules on the arrays may be oligonucleotides, DNA fragments, or hybridizing analogues or mimetics thereof, whose sequences are derived from known genes of the physiological source being analyzed. The locations of the polymeric molecules are annotated so that hybridization signals on the spots may be correlated to expression of a particular gene in the physiological source. Of particular interest of this subject invention are the arrays of immobilized polymeric nucleic acid molecules corresponding to a particular subset of the total genes expressed in physiological source, for example, cDNA clones according to their roles in specific biological pathways such as cell cycle, apoptosis, immune response, and cancer, etc.

The supporting surface for the nucleic acid array may be fabricated from a variety of materials, such as plastics, ceramics, silicon, metals, gels, membranes, glasses, and the like.

A variety of different methodologies have been developed for producing nucleic acid arrays. Representative methodologies include spotting methods, in which probes are immobilized or spotted on the surface of substrates as described in WO 95/35505 the disclosure of which is herein incorporated by reference, and methods in which the cDNA/oligonucleotide fragments are synthesized or grown on the surface of the substrates, such as U.S. Pat. No. 5,445,934, the disclosures of which are herein incorporated by reference. Arrays of cDNA/oligonucleotide fragments spotted onto nylon membranes are described by Lennon G G and Lerach H in Trends in Genetics (1991) 7:314-317; Gress et al. in Mammalian Genome (1992) 3:609-619; Meier-Ewert et al. in Nature (1993) 361:375-376; Nguyen et al. in Genomics (1995) 29:207-216; Zhao et al. in Gene (1995) 156:207-213; Takahashi et al. in Gene (1995) 164:219-217; Milosavlijevic et al. in Genome Research (1996) 6:132-141; Pietu et al. in Genome Research (1996) 6:492-503; and Drmanac R et al. in Science (1993) 260:1649-1652. Arrays of cDNA/oligonucleotide fragments spotted onto the surface of modified microscope glass slides are described by Shena et al. in Science (1995) 270: 467-470 and Shalon et al. in Genome Research (1996) 6: 639-645. Arrays in which the cDNA/oligonucleotide fragments have been grown on the surface of a substrate are described by Lockhart et al. in Nature Biotechnology (1996) 14:1675.

Of particular interest for use in the analysis of differential gene expression in human and mouse physiological sources are the arrays of subsets human and mouse cDNAs sold under the trademark GEArray™ by SuperArray Bioscience Corporation (Frederick, Md.).

F. Kits

The invention also provides kits for use in carrying out the subject methods, e.g. generating populations of labeled nucleic acids, performing differential gene expression analysis and the like. The kits according to the subject invention include at least the set of gene specific primers that are employed to generate the labeled oligonucleotides. The gene-specific primers in the kits may have at least 20, usually at least 50 and more usually at least 100 of the gene specific primers. The kits may further comprise one or more additional reagents employed in the various methods, dNTPs and/or rNTPs, which may be either premixed or separate, one or more uniquely labeled dNTPs and/or rNTPs, such as biotinylated or Cy3 or Cy5 tagged dNTPs, enzymes, such as reverse transcriptases, DNA polymerases (e.g., Taq DNA polymerase) and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like. The kits may also comprise instructions for performing any of the methods described herein.

The following examples are offered by way of illustration and not by way of limitation.

### G. EXAMPLES

### Example 1

### Generation of Biotinated Second-Strand cDNA Probe from Total RNA by LPR

1. RT Reaction to Synthesize First Strand cDNA

Total RNA from a sample was reversed transcribed to generate first strand cDNA. Total RNA was mixed in a sterile PCR tube with 2.5 μg of total RNA, 1 μl of Buffer P (random hexamers at 1 μg/μl, Promega Cat# M1701), and RNase-free H₂O to adjust the final volume to 10 μl. The contents were gently mixed with a pipettor followed by brief centrifugation. The mixture was placed in a thermal cycler at 70° C. for 3 min, and then cooled to 37° C. and kept at that temperature for 10 min. The Annealing Mixture was prepared.

A 10 μl RT Cocktail was prepared by mixing the following: 4 μl of RNase-free H₂O, 4 μl of Buffer BN (5× GEAlabeling Buffer containing 250 mM Tris HCl, pH 8.5, 200 mM KCl, 40 mM MgCl₂, 10 mM DTT, 2.5 mM dATP, 2.5 mM dGTP, 2.5 mM dCTP, 0.25 mM dTTP), 1 μl of RNase Inhibitor (RI, 40 u/μl, Promega Cat# N2511), and 1 μl of Reverse Transcriptase (RE, M-MLV Reverse Transcriptase at 200 u/μl, Promega Cat# M1701). The RT Cocktail was incubated at 37° C. for 1 min before proceeding to the next step.

For each array, 10 μl of the RT Cocktail was mixed gently with 10 μl of the Annealing Mixture using a pipettor, and incubated at 37° C. for 25 min. RNA was hydrolyzed and the reverse transcriptase was inactivated by incubating at 85° C. for 5 min. The finished RT Reaction was kept on ice until the next step.

2. LPR Labeling Reaction

A 30 μl LPR Cocktail was prepared by mixing the following in a sterile microcentrifuge tube: 18 μl of Buffer L (1×LPR Buffer, 10 mM Tris HCl, 50 mM KCl, 1.5 mM MgCl₂), 9 μl of Buffer AF (mix of representational number of gene-specific primers (0.3 μM of each individual primer), each primer has a sequence identical to a sequence in a distinct mRNA molecule), 2 μl of Biotin-16-dUTP (1 mM, Roche, Cat. No. 1-093-070), and 1 μl of DNA Polymerase (LPR DNA Polymerase: Taq DNA Polymerase, 5 u/μl, Promega Cat# M1661).

To each finished RT reaction, 30 μl of the LPR Cocktail was added and mixed gently with a pipettor. LPR reaction was performed with the thermal cycler as follows: 85° C., 5 min; 30 cycles of (85° C., 1 min; 50° C., 1 min; 72° C., 1 min); then 72° C., 5 min. LPR reaction was immediately stopped by adding 5 μl of Buffer C (10× Stop Solution: 100 mM EDTA) and chilled on ice. The labeled cDNA probes generated by LPR reaction were denatured by heating at 94° C. for 2 min, and chilled quickly on ice. The labeled cDNA probes were then ready for hybridization.

### Example 2

### Generation of Biotinated Second-Strand cDNA Probe from Anti-Sense RNA (aRNA) by LPR

RT reaction was performed as described in Example 1 except that 2.5 μg of total anti-sense RNA were used instead of total RNA to synthesize first strand cDNA. LPR labeling reaction was performed as described in Example 1 except that 9 μl of Buffer A (mix of representational number of gene-specific primers (0.3 μM of each individual primer), each primer has a sequence complementary to a sequence in a distinct mRNA molecule) were used instead of Buffer AF to generate biotinated second-strand cDNA probes.

### Example 3

### Generation of cDNA Fragments and Immobilization of the Fragments on Nylon Membrane

For each array, 100 cDNA fragments corresponding 100 different human genes were amplified by RT-PCR from human total RNA of various cell and tissue origins using a combination of sense and antisense gene-specific primers. Among those 100 genes, 96 genes were reported involved in a defined biological pathway, and 4 genes were so-called “housekeeping” genes that were used for controls between experimental samples.

3.1 RT Reaction

For each total RNA sample, the following were mixed in a sterile PCR tube: 2.5 μg of RNA, 1 μl of Buffer P as described in Example 1, 1 μl of Oligo dT₂₀ (1 μg/μl oligonucleotide of 20 dTTP, synthesized by Integrated DNA Technologies, Inc., Coralville, Iowa), and RNase-free H₂O to adjust the final volume to 10 μl. The contents were gently mixed with a pipettor followed by brief centrifugation. The mixture was placed in a thermal cycler at 70° C. for 3 min, and then cooled to 37° C. and kept at that temperature for 15 min. The Annealing Mixture was prepared.

A 10 μl RT Cocktail was prepared by mixing the following: 4 μl of RNase-free H₂O, 4 μl of dNTPs Mix (2.5 mM of each of the 4 dNTPs: dATP, dGTP, dCTP and dTTP), 1 μl of RNase Inhibitor (RI, 40 u/μl, Promega Cat# N2511), and 1 μl of Reverse Transcriptase (RE, M-MLV Reverse Transcriptase at 200 u/μl, Promega Cat# M1701). The RT Cocktail was incubated at 37° C. for 1 min before proceeding to the next step.

For each array, 10 μl of the RT Cocktail was mixed gently with 10 μl of the Annealing Mixture using a pipettor, and incubated at 37° C. for 25 min. RNA was hydrolyzed and the reverse transcriptase was inactivated by incubating at 85° C. for 5 min. The finished RT Reaction was kept on ice until the next step.

3.2 PCR Amplification of cDNA Fragments

One tenth of the volume of the above finished RT reaction was used as template, and the PCR mix was set up as follows: 2 μl of Template (RT reaction products), 45 μl of Buffer L (1×LPR Buffer, 10 mM Tris HCl, 50 mM KCl, 1.5 mM MgCl₂), 2 μl of Primer pair 1 (5 μM of gene-specific primer pairs designed to produce gene-specific cDNA fragments of 300 bp to 600 bp), 0.5 μl of dNTP Mix (2.5 mM of each of the 4 dNTPs: dATP, dGTP, dCTP and dTTP), and 0.5 μl of DNA Polymerase (LE, Taq DNA Polymerase, 5 u/μl, Promega Cat# M1661). The PCR reaction was performed with the thermal cycle as follows: 94° C. 5 min and 30 cycles of (94° C., 15 sec; 50° C., 15 sec; 72° C., 15 sec); then 72° C., 10 min. Then, the PCR products were examined on 1% agarose/EtBr gels in 1×TBE buffer. A 100 bp DNA Ladder was used as a DNA size marker.

3.3 TA Cloning of the PCR Fragments

If the size of the PCR product agreed with the predicted size, the PCR fragment was cloned into the vector pCRII (Invitrogen) or pGEM-T (Promega) according to the manufacture's instruction. After individual clone with the insert was identified, another PCR reaction with a set of nested primers (Primer pair 2, which are nested within Primer pair 1) was used to confirm the sequence, of the insert.

3.4 PCR Amplification of Cloned Gene-Specific Fragments

To confirm the sequence of the insert, the following PCR mix was set up (50 μl reaction volume): 2 μl of Plasmid template (10 ng/μl), 45 μl of Buffer L (10 mM Tris HCl, 50 mM KCl, 1.5 mM MgCl₂), 2 μl of Primer pair 2 (5 μM of gene-specific primer pairs), 0.5 μl of dNTP Mix (2.5 mM of each of the 4 dNTPs: dATP, dGTP, dCTP and dTTP), and 0.25 μl of DNA Polymerase (LE, Taq DNA Polymerase, 5 u/μl, Promega Cat#. M1661). The PCR reaction was performed with the thermal cycle as follows: 94° C. 5 min and 35 cycles of (94° C., 20 sec; 50° C., 20 sec; 72° C., 30 sec). Then, the PCR products were precipitated by adding 70% volume of isopropanol and centrifuged at 14,000 rpm in a microcentrifuge for 20 min. The DNA pellet was the resuspended in deionized water and a portion of it was examined on 1% agarose/EtBr gels in 1×TBE buffer. A 500 bp DNA fragment was used as a DNA mass marker, the concentration of which had been calibrated as 100 ng/μl. Then the concentrations of all the cDNA fragments were adjusted to 100 ng/μl accordingly.

3.5. cDNA Array Printing

In each well of a 96-well plate, 200 μl of each individually calibrated cDNA solution (100 ng/μl) was mixed with 15 μl of BPB dye (0.01%) and 20 μl of NaOH (1 M). 15 nl of this mixture was then deposited on the positively charged nylon membrane (Schleher & Schull) using the ink-jet, non-contact nanoliter (nl) dispensing system (the synQUAD™ technology) from Cartesian Technologies (a Genomic Solutions company). A separate 96-well souce plate with 4 housekeeping genes and negative control DNAs was used in the second round of printing to complete the printing of GEArray™ Q-series cDNA arrays (GEArray™, SuperArray Bioscience Corporation).

### Example 4

### Hybridization Biotin-Labeled cDNA Probe with cDNA Array and Chemiluminescent Detection

1. Pre-Hybridization

The array membrane was pre-wetted by adding roughly 5 ml of deionized water to the hybridization tube. The tube was allowed to sit inverted while the GEAprehyb was prepared.

GEAhyb (1× hybridization solution, 5×SSPE, 10× Denhardt's, 2% SDS) was heated to 60° C., and the bottle containing the GEAhyb was inverted several times to allow complete dissolution of the buffer components. The sheared salmon sperm DNA was heated at 100° C. for 5 min and was then immediately chilled on ice. To prepare GEAprehyb, the heat-denatured salmon sperm DNA was added to the pre-warmed GEAhyb to a final concentration of 100 μg/ml. GEAprehyb solution was kept at 60° C. until needed.

The deionized water was discarded from the hybridization tube. 2 ml of the GEAprehyb solution was added into the hybridization tube, and the tube was gently vortexed for a few seconds. The cap of the tube was screwed on hand-tight. The tube was placed inside a hybridization cylinder. Two GEArray Q or S Series hybridization tubes would fit inside a standard hybridization cylinder (ID×L=35×150 mm). Pre-hybridize was performed in a hybridization oven at 60° C. for 1 to 2 hours with continuous agitation at 5 to 10 rpm.

2. Hybridization

To prepare for hybridization, the entire volume of denatured cDNA probes (generated in Example 1 or 2) were added to 0.75 ml of pre-warmed GEAprehyb. The solution (GEAhyb containing probes) was mixed well and kept at 60° C. The GEAprehyb from the hybridization tube was discarded. GEAhyb containing probes were added to the hybridization tube. Hybridization was performed overnight at 60° C. with continuous agitation at 5 to 10 rpm.

3. Washing

Excess Wash Solution 1 (2×SSC, 1% SDS) and Wash Solution 2 (0.1×SSC, 0.5% SDS) were prepared and warmed to 60° C. GEAhyb solution containing probes was discarded from the hybridization tube. The membrane was washed twice with 5 ml Wash Solution 1 (2×SSC, 1% SDS) 60° C. with 20 to 30 rpm agitation for 15 minutes each. The tube was vortexed gently with each wash. The membrane was washed twice with 5 ml Wash Solution 2 (0.1×SSC, 0.5% SDS) at 60° C. with 20 to 30 rpm agitation for 15 minutes each. The tube was vortexed gently with each wash.

4. Chemiluminescent Detection

GEAblocking solution (0.2% Gelatin in 1× Detector Block Solution, KPL, Cat# 71-83-03, Kirkegaard & Perry Laboratories, Inc., Gaithersburg, Md.) and 5× Buffer F (250 mM Tris HCl pH 7.5, 750 mM NaCl, 1.25% SDS) were warmed to 37° C. and the bottles containing the solutions were inverted several times to allow any precipitate to completely dissolve. The solutions were kept at room temperature until needed.

1) Blocking the Array

After the last wash was discarded from the hybridization tube, 2 ml GEAblocking solution was immediately added into the tube. The tube was incubated for 40 min with continuous agitation at 20 to 30 rpm.

2) Binding of Alkaline Phosphatase-Conjugated Streptividin (AP)

A Binding Buffer was prepared by diluting 5× Buffer F (250 mM Tris HCl pH 7.5, 750 mM NaCl, 1.25% SDS) five-fold, and then diluting alkaline phosphatase-conjugated streptividin (KLP Cat# 475-3000) at 1:7,500 into 1× Buffer F. GEAblocking solution was discarded from the hybridization tube. 2 ml of the Binding Buffer was added to the hybridization tube, and the hybridization tube was incubated for 10 min with continuous but gentle (5-10 rpm) agitation.

3) Washing

The membrane was washed four times with 4 ml 1× Buffer F for 5 min with gentle agitation. The tube was gently vortexed after each addition of fresh 1× Buffer F. The membrane was then rinsed and washed twice with 3 ml Buffer G (100 mM Tris HCl pH 9.5, 100 mM NaCl, 10 mM MgCl₂).

4) Detection

1.0 ml CDP-Star chemiluminescent substrate (KLP Cat# 50-60-05) was added into the hybridization tube. The hybridization tube was incubate at room temperature for 2 to 5 min. Alternatively, the GEArray Q membrane could be placed on a sheet of plastic wrap and the 1.0 ml of CDP-Star solution was dropped onto the membrane. The membrane was blotted on a piece of filter paper to remove excess CDP-Star Solution. The membrane was placed between two plastic sheets or into a small plastic zip-lock bag, and bubbles were smoothed out.

5) Image Acquisition, Data Acquisition and Analysis

The membrane was exposed to a CCD camera for 15 min at the Alpha Imager Station (Alpha Innotech Corporation, San Leandro, Calif.) to acquire the image. Fluor Chem v2.0 Stand Alone and the Alpha Imager accompanying software were used to digitize the image and the data were exported to a spreadsheet in Microsoft Excel and analyzed by GEArray Analyzer, a free data analysis software from SuperArray (www.superarray.com).

Each GEArray™ Q Series membrane was spotted with a negative control of pUC18 DNA, blanks, and housekeeping genes, including, β-actin, GAPDH, cyclophilin A and ribosomal protein L13a. All raw signal intensities were corrected for background by subtracting the signal intensity of a negative control or blank. All signal intensities were normalized to that of a housekeeping gene. These corrected, normalized signals were then used to estimate the relative abundance of particular transcripts.

### Example 5

### Comparison Between RT-Labeled Probes and the LPR Labeled Probes

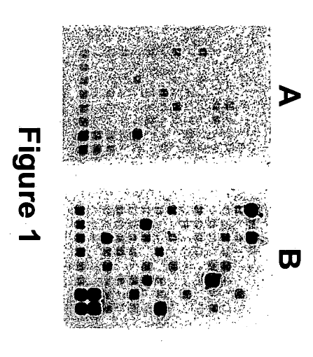

Human Universal Reference RNA (2.5 μg, BD Biosciences, Clontech Cat# 636538) was used to synthesize biotin-labeled probes using either MMLV reverse transcriptase or LPR method described in Example 1. Gene-specific primer set (Buffer A-HS023 of SuperArray Cat# HS-023N) was used to synthesize biotin-labeled first-strand cDNA with MMLV reverse transcriptase (Promega Cat# M1701) according to the reaction condition recommended by Promega. Gene-specific primer set (Buffer AF-HS023 of SuperArray Cat# HS-023N) was used to synthesize biotin-labeled second-strand cDNA with Taq DNA polymerase (Promega Cat# M2661) for 30 cycles according to LPR procedure as described in Example 1. The labeled probes generated from both methods were hybridized to separate membranes from the GEArray™ Q Series Human TGFβ/BMP Signaling Pathway Kit (SuperArray, Cat# HS-023N), generated according to Example 3. Signals were detected using the Chemiluminescent Detection Method. As shown in FIG. 1, the probes generated by the RT method with MMLV reverse transcriptase (FIG. 1A) had much higher background and much lower real signal intensities than the ones generated by LPR method (FIG. 1B).

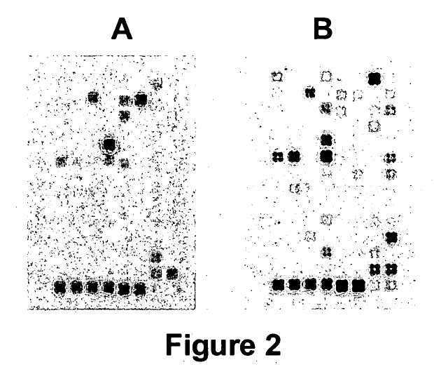

FIG. 2 shows a comparison of hybridization patterns using probes generated by RT method and LPR method from aRNA. 0.05 μg of human brain total RNA (BD Biosciences, Clontech Cat# 636530) was amplified two round with RiboAmp™ OA RNA Amplification Kit (Arcturus, Cat# KIT0206) according to the manufacturer's instruction. 3.0 μg of amplified aRNA was labeled either by MMLV reverse transcriptase as described above or LPR method described in Example 2. Gene-specific primer set (Buffer A-HS018 of SuperArray Cat#HS-018) was used with MMLV reverse transcriptase as described above; and gene-specific primer set (Buffer AF-HS018 of SuperArray Cat#HS-018) was used with LPR method as described in Example 2. The labeled nucleic acids using RT method and LPR method were separately hybridized to GEArray Q-series Human Neurotrophin and Receptors Gene Array (SuperArray, Cat#HS-018) generated according to Example 3. As shown in FIG. 2, hybridizing with labeled probes generated by LPR labeling method (FIG. 2B) had better signal intensities over background than hybridizing with labeled probes generated by the RT method with MMLV reverse transcriptase (FIG. 2A).

To demonstrate that the gene expression profile generated by LPR method is more reliable than that generated by conventional labeling method, mouse liver or mouse thymus total RNA (7 and 5 μg, respectively, BD Biosciences) was used to synthesize biotin-labeled probe using either MMLV reverse transcriptase as described above or LPR method as described in Example 1, and hybridized to GEArray™ Q-series Mouse Insulin Signal Pathway Kit (SuperArray, Cat# MM-030; array produced according to Example 3). Signals were then detected using the Chemiluminescent Method. Sequences of gene-specific primer set (Buffer A-MM030 of SuperArray Cat# MM-030) was used with MMLV reverse transcriptase. Gene-specific primer set (Buffer AF-MM030 of SuperArray Cat# MM-030) was used with LPR method as described in Example 1. Mouse liver or mouse thymus total RNA were used to generate a RT-PCR profile according to the method described in Step 3.1-3.2 of Example 3. The PCR products were examined on 1% agarose/EtBr gels in 1×TBE buffer. Then each band of the PCR products was rearranged with Adobe Photoshop to match the location of each individual gene spotted on the Mouse Insulin Signaling Pathway Array. “Primer pair 1” used in each PCR reaction was the two corresponding primers from Buffer A-MM030 and Buffer AF-MM030. As shown in FIG. 3, the gene expression profile generated by LPR probes (FIG. 3B) was much closer to the profile generated by RT-PCR (FIG. 3C) compared with the probes generated by RT method (FIG. 3A).

1. A method of producing a sub-population of labeled nucleic acids, said method comprising:
(a) synthesizing first strand cDNA from a sample of RNA through reverse transcription, wherein the sample of RNA is obtained from a physiological source;
(b) contacting said first strand cDNA with a pool of a representational number of at least 15 distinct gene specific primers under conditions that allow formation of hybrid duplexes between said gene specific primers and said first strand cDNA, wherein each constituent gene specific primer has a sequence complementary to a distinct first strand cDNA; and
(c) enzymatically extending said gene specific primers from said hybrid duplexes to generate a sub-population of labeled nucleic acids.

2. The method of claim 1, wherein said sub-population of labeled nucleic acids extended from said gene specific primers is generated through a single cycle of unidirectional DNA polymerization.

3. The method of claim 1, wherein said sub-population of labeled nucleic acids extended from said gene specific primers is generated through multiple cycles of uni-directional DNA polymerization.

4. The method of claim 1, wherein said sample of RNA comprises total RNA.

5. The method of claim 1, wherein said sample of RNA comprises mRNA.

6. The method of claim 1, wherein said sample of RNA comprises amplified antisense RNA (aRNA).

7. The method of claim 1, wherein said first strand cDNA is synthesized through RNA self-priming without addition of any exogenous synthetic primers.

8. The method of claim 1, wherein said first strand cDNA is synthesized through addition of synthetic random primers.

9. The method of claim 1, wherein said first strand cDNA is synthesized through addition of oligo dT primers.

10. The method of claim 1, wherein said pool of gene specific primers comprises at least 20 distinct gene specific primers.

11. The method of claim 1, wherein said pool of gene specific primers comprises at least 50 distinct gene specific primers.

12. The method of claim 1, wherein said pool of gene specific primers comprises one oligonucleotide sequence for a single gene.

13. The method of claim 1, wherein said pool of gene specific primers comprises more than one oligonucleotide sequences for a single gene.

14. The method of claim 1, wherein the said label is directly detectable.

15. The method of claim 1, wherein the said label is detectable after a subsequent chemical or enzymatic reaction.

16. A method of producing a sub-population of labeled nucleic acids, said method comprising:
(a) synthesizing first strand cDNA from a sample of RNA through reverse transcription, wherein the sample of RNA is obtained from a physiological source;
(b) generating a sub-population of labeled nucleic acids using polymerase chain reaction (PCR) with a pool of a representational number of at least 15 pairs of distinct gene specific primers, wherein one gene specific primer in each pair comprises a sequence complementary to the sense sequence of said distinct gene, and the other primer in the pair comprises a sequence complementary to the antisense sequence of said distinct gene.

17. The method of claim 16, wherein said PCR is an asymmetric PCR.

18. The method of claim 16, wherein said PCR is performed in one cycle.

19. The method of claim 16, wherein said PCR is performed in multiple cycles.

20. The method of claim 16, wherein said sample of RNA comprises total RNA.

21. The method of claim 16, wherein said sample of RNA comprises mRNA.

22. The method of claim 16, wherein said sample of RNA comprises amplified antisense RNA (aRNA).

23. The method of claim 16, wherein said first strand cDNA is synthesized through RNA self-priming without addition of any exogenous synthetic primers.

24. The method of claim 16, wherein said first strand cDNA is synthesized through addition of synthetic random primers.

25. The method of claim 16, wherein said first strand cDNA is synthesized through addition of oligo dT primers.

26. The method of claim 16, wherein said pool of gene specific primers comprises at least 20 distinct gene specific primers.

27. The method of claim 16, wherein said pool of gene specific primers comprises at least 50 distinct gene specific primers.

28. The method of claim 16, wherein the said label is directly detectable.

29. The method of claim 16, wherein the said label is detectable after a subsequent chemical or enzymatic reaction.

30. A method of analyzing the differences in the expression pattern of the genes of special interest between a plurality of different physiological samples, said method comprising:
(a) synthesizing first strand cDNA from a sample of RNA through reverse transcription, wherein the sample of RNA is obtained from a physiological source;
(b) contacting a pool of a representational number of at least 15 distinct gene specific primers with said first strand cDNA under conditions that allow formation of hybrid duplexes between said gene specific primers and said first strand cDNA, wherein each constituent gene specific primer has a sequence complementary to a distinct first strand cDNA; and
(c) enzymatically extending said gene specific primers from said hybrid duplexes to generate a sub-population of labeled nucleic acids and
(d) comparing the populations of labeled nucleic acids from each physiological source to identify the differences in the populations.

31. The method of claim 30, wherein the comparing step comprises:
hybridizing the labeled nucleic acids from each of the distinct physiological samples to an array of nucleic acids stably associated with the surface of a substrate;
washing off the unbound labeled nucleic acids from the surface to produce a detectable hybridization patterns for each of the distinct physiological samples; and
comparing the hybridization patterns for each of the distinct physiological samples.

32. A method of analyzing the differences in the expression pattern of the genes of special interest between a plurality of different physiological samples, said method comprising:
(a) synthesizing first strand cDNA from a sample of RNA through reverse transcription, wherein the sample of RNA is obtained from a physiological source;
(b) generating a sub-population of labeled nucleic acids using polymerase chain reaction (PCR) with a pool of a representational number of at least 15 pairs of distinct gene specific primers, wherein one gene specific primer in each pair comprises a sequence complementary to the sense sequence of said distinct gene, and the other primer in the pair comprises a sequence complementary to the antisense sequence; and
(c) comparing the populations of labeled nucleic acids from each physiological source to identify the differences in the populations.

33. The method of claim 32, wherein the comparing step comprises:
hybridizing the labeled nucleic acids from each of the distinct physiological samples to an array of nucleic acids stably associated with the surface of a substrate;
washing off the unbound labeled nucleic acids from the surface to produce a detectable hybridization patterns for each of the distinct physiological samples; and
comparing the hybridization patterns for each of the distinct physiological samples.

34. The method of claim 32, wherein said PCR is an asymmetric PCR.

35. The method of claim 32, wherein said PCR is performed in multiple cycles.
